(19) United States
(12) Patent Application Publication
(10) Pub. No.: US 2019/0059811 A1
(43) Pub. Date: Feb. 28, 2019

(54) COMBINATION PEAK FLOW METER, INHALER, AND NEBULIZER

(71) Applicant: Charles Dashiell, Severn, MD (US)

(72) Inventor: Charles Dashiell, Severn, MD (US)

(21) Appl. No.: 15/687,508

(22) Filed: Aug. 27, 2017

(51) Int. Cl. A61B 5/00; A61B 5/087; A61B 5/097; A61M 15/00; A61M 11/02; A61M 16/14

(52) U.S. Cl. CPC A61B 5/4839; A61B 5/0871; A61B 5/097; A61M 15/009; A61B 2560/0443; A61M 11/02; A61M 16/14; A61M 2205/583; A61M 15/0021

(57) ABSTRACT

A combination device, including a body, a mouthpiece connected to a first end of the body, an inhaler connected to a second end of the body, a peak flow meter connected to the body between the mouthpiece and the inhaler, and a nebulizer connected to a lateral side portion of the body.

### BACKGROUND OF THE INVENTION

### 1. Field of the Invention

The present general inventive concept relates generally to a combination peak flow meter, inhaler, and nebulizer.

### 2. Description of the Related Art

A conventional peak flow meter is an inexpensive, portable, handheld device for those with asthma that is used to measure how well air moves out of a user's lungs. Measuring the user's peak flow using this meter is an important part of managing asthma symptoms and preventing an asthma attack.

A conventional inhaler (or puffer) is a medical device used for delivering medication into the user's body via the lungs. It is mainly used in the treatment of asthma and chronic obstructive pulmonary disease. To reduce deposition in the mouth and throat, and to reduce the need for precise synchronization of the start of inhalation with actuation of the device, metered dose inhalers (MDIs) are sometimes used with a complementary spacer or holding chamber device.

A conventional nebulizer is drug delivery device used to administer medication in the form of a mist inhaled into lungs of the user. Nebulizers use oxygen, compressed air or ultrasonic power to break up solutions and suspensions into small aerosol droplets that can be directly inhaled from the mouthpiece of the device. More specifically, nebulization utilizes the venturi principle to fracture liquid medications into a fine aerosol mist, which a patient inhales, thereby absorbing the active ingredients of the aerosol across the pulmonary membranes to effect the desired treatment. It is a standard strategy for the therapeutic application of medicinal aerosols for a wide range of pulmonary dysfunctions, including asthma, COPD, bronchitis, cystic fibrosis, croup, etc. The conventional nebulizer is often connected to a machine and/or apparatus that produces a cloud of medication by passing a jet of compressed air though a solution of a drug, or by dropping the drug solution onto a plate vibrating at a high frequency.

However, there is a need for a combination device that combines a peak flow meter, an inhaler, and a nebulizer, to add convenience and universality for a user that has pulmonary conditions.

### SUMMARY

The present general inventive concept provides a device that is combination of a peak flow meter, an inhaler, and a nebulizer.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a combination device, including a body, a mouthpiece connected to a first end of the body, an inhaler connected to a second end of the body, a peak flow meter connected to the body between the mouthpiece and the inhaler, and a nebulizer connected to a lateral side portion of the body.

The nebulizer may include a nebulizer body having a connector to connect to the body of the combination device, a dispenser to expel medicine therefrom to the body of the combination device, and a tubing to connect the dispenser to the nebulizer body.

The dispenser may include an apparatus to automatically expel medicine and air therefrom.

The inhaler may include a canister containing medicine therein, a casing to house the canister therein, and an inhaler mouthpiece to connect to the body of the combination device.

The depressing of the canister within the casing may propel the medicine through the inhaler mouthpiece towards and through the mouthpiece of the combination device.

The peak flow meter may include a numbered scale including numbers representing a peak expiratory flow, an indicator to indicate a user's peak expiratory flow according to the numbered scale, and an indicator groove to allow the indicator to slide along to indicate the user's peak expiratory flow as represented on the numbered scale.

At least one of the mouthpiece, the inhaler, the peak flow meter, and the nebulizer may be detachably connected to the body of the combination device.

### BRIEF DESCRIPTION THE DRAWINGS

These and/or other features and utilities of the present general inventive concept will become apparent and more readily appreciated from the following description of the embodiments, taken in conjunction with the accompanying drawings of which:

FIG. 1 is view of a combination device, according to an exemplary embodiment of the present general inventive concept.

### DETAILED DESCRIPTION OF THE INVENTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

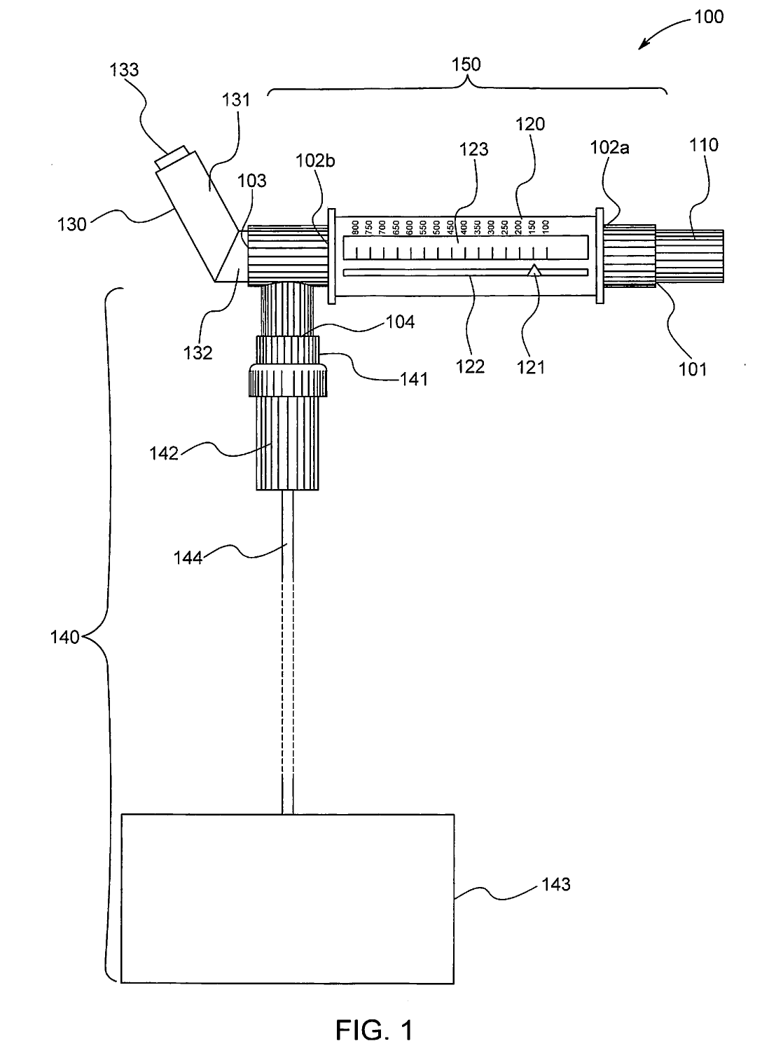

FIG. 1 is view of a combination device 100, according to an exemplary embodiment of the present general it concept.

The combination device 100, and all components included therewith, may be made from BPA-free plastic, metal, rubber, ceramic, or any other type of material known to one of ordinary skill in the art.

Referring to FIG. 1, the combination device 100 may include a mouthpiece 110, a peak flow meter 120, an inhaler 130, a nebulizer 140, a body 150, a mouthpiece connecting portion 101, peak flow meter connecting portions 102a and 102b, an inhaler connecting portion 103, and a nebulizer connecting portion 104.

The body 150 may be cylindrical, octagonal, rectangular, or have any other shape.

The mouthpiece 110 may be connected to the body 150 at a first end of the body 150.

The inhaler 130 may be connected to the body 150 at a second end of the body 150.

The peak flow meter 120 may be connected to the body 150 between the mouthpiece 110 and the inhaler 120. The peak flow meter 120 may be a permanent part of the body 150, or may alternatively be disconnecting from the body 150.

The nebulizer 140 may be connected to the body 150 at a lateral side portion of the body 150.

Alternatively, the mouthpiece 110, the peak flow meter 120, the inhaler 130, and the nebulizer 140 may be connected at any portions of the body 150 of the combination device 100.

Furthermore, the mouthpiece 110, the peak flow meter 120, the inhaler 130, and the nebulizer 140 may be permanently connected at any portions of the body 150 of the combination device 100, or alternatively, may be detachably connected at any portions of the body 150 of the combination device 100

Referring to FIG. 1, the mouthpiece 110 may be connected to the body 150 of the combination device 100 using the mouthpiece connecting portion 101. The mouthpiece connecting portion 101 may be a hole, a screw-on device, a rod, a snap, or any other type of connector to allow the mouthpiece 110 to be interchanged such that other mouth pieces may be attached to the combination device 100.

A first end of the mouthpiece 110 may be connected to the body 150, while a second end of the mouthpiece 110 may be inserted into a mouth of a user to allow the user to utilize any or all of the peak flow meter 120, the inhaler 130, and/or the nebulizer 140. In other words, the mouthpiece 110 may allow the user to inhale or exhale therethrough (i.e., suck or blow).

Referring to FIG. 1, the peak flow meter 120 may be a device that measures how fast air comes out of the lungs when the user exhales forcefully after inhaling fully. This measure is called a “peak expiratory flow,” or “PEF.”

The peak flow meter 120 may include an indicator 121, and indicator groove, and a numbered scale 123.

The peak flow meter 120 may be connected to one portion of the body 150 of the combination device 100 using the peak flow meter connecting portion 102a, and may be connected to another portion of the body 150 of the combination device 100 using the peak flow meter connecting portion 102b. The peak flow meter connecting portions 102a and 102b may be holes, screw-on devices, a rod, a snap, or any other type of connectors to allow the peak flow meter 120 to be interchanged such that other peak flow meters may be attached to the combination device 100.

To use the peak flow meter 120 of the combination device 100, the user may stand up or sit up straight, making sure that the indicator 121 is at a bottom (i.e., at a zero point) of the numbered scale 123. The user then may inhale deeply though the mouth, thereby filling his/her lungs completely, then the user may place the mouth piece 110 in his/her mouth, after which the user may blast air out the mouth as hard as possible in a single blow. After the user blasts the air out, the force of the air may cause the indicator 121 to slide along the indicator groove 122, and thereby move to a new position corresponding to the numbered scale 123, thereby indicating a PEF of the user. The further the indicator 121 moves along the indicator groove 122 corresponding to numbered scale 123, the better the user's PEF. The indicator 121 may be manually reset along the indicator groove 122 to correspond to a zero position of the numbered scale 123, after the user finishes reading the numbered scale 123.

Referring to FIG. 1, the inhaler 130 may include a casing 131, an inhaler mouthpiece 132, and a canister 133.

The canister 133 may be inserted within the casing 131, such that medicine within the canister 133 is expelled out the inhaler mouthpiece 132 when the canister is depressed down within the casing 131.

The inhaler mouthpiece 132 may be connected to the body 150 of the combination device 100 using the inhaler connecting portion 103. The inhaler connecting portion 103 may be a hole, a screw-on device, a rod, a snap, or any other type of connector to allow the 130 to be interchanged such that other inhalers may be attached to the combination device 100.

Referring to FIG. 1, the nebulizer 140 may include a connector 141, a nebulizer body 142, a dispenser 143, and a tubing 144.

The connector 141 may be connected to the body 150 of the combination device 100 using the nebulizer connecting portion 101. The nebulizer connecting portion 101 may be a hole, a screw-on device, or any other type of connector to allow the nebulizer 140 to be interchanged such that other nebulizers may be attached to the combination device 100.

The nebulizer body 142 of the nebulizer 140 may have the tubing 144 attached thereto, in order to allow the dispenser 143 to be connected to the combination device 100. As such, the dispenser 143 may be connected to the nebulizer body 142 via the tubing 144.

The dispenser 143 may be a dispenser containing medicine, and may be hand-operated, or alternatively, may be a machine and/or apparatus that propels medicine and air through the tubing 144 to the combination device 100. If the dispenser 143 is embodied as a machine and/or apparatus, it may be plugged in to an electrical outlet, or may be battery-operated.

The nebulizer 140 may use oxygen, compressed air or ultrasonic power to break up solutions and suspensions into small aerosol droplets (i.e., a mixture of gas and solid or liquid particles), to allow the user to inhale the solution through the mouthpiece 110 of the combination device 100. As such, the user may receive medicine from the dispenser 143 via the mouthpiece 110.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

What is claimed is:

1. A combination device, comprising:
a body;
a mouthpiece connected to a first end of the body;
an inhaler connected to a second end of the body;
a peak flow meter connected to the body between the mouthpiece and the inhaler; and
a nebulizer connected to a lateral side portion of the body.

2. The combination device of claim 1, wherein the nebulizer comprises:
a nebulizer body having a connector to connect to the body of the combination device;
a dispenser to expel medicine therefrom to the body of the combination device; and
a tubing to connect the dispenser to the nebulizer body.

3. The combination device of claim 2, wherein the dispenser comprises an apparatus to automatically expel medicine and air therefrom.

4. The combination device of claim 1, wherein the inhaler comprises:
a canister containing medicine therein;
a casing to house the canister therein; and
an inhaler mouthpiece to connect to the body of the combination device.

5. The combination device of claim 4, wherein depressing the canister within the casing propels the medicine through the inhaler mouthpiece towards and through the mouthpiece of the combination device.

6. The combination device of claim 1, wherein the peak flow meter comprises:
a numbered scale including numbers representing a peak expiratory flow;
an indicator to indicate a user's peak expiratory flow according to the numbered scale; and
an indicator groove to allow the indicator to slide along to indicate the user's peak expiratory flow as represented on the numbered scale.

7. The combination device of claim 1, wherein at least one of the mouthpiece, the inhaler, the peak flow meter, and the nebulizer is detachably connected to the body of the combination device.
